(19) United States
(12) Patent Application Publication
(10) Pub. No.: US 2003/0207932 A1
(43) Pub. Date: Nov. 6, 2003

(54) Compositions that prevent post-traumatic hyperpigmentation and methods related thereto

(76) Inventor: Morris Mann, Glendale, AZ (US)

Correspondence Address: The Halvorson Law Firm, 405 W. Southern Ave., Ste 1, Tempe, AZ, 85282, US

(21) Appl. No.: 10/428,409

(22) Filed: May 1, 2003

Related U.S. Application Data
(60) Provisional application No. 60/377,857, filed on May 3, 2002.

(51) Int. Cl. A61K 31/47; A61K 31/405; A61K 7/135; A61K 31/045

(52) U.S. Cl. 514/419; 514/729; 514/311

(57) ABSTRACT

The ingestion or topical administration of substances that inhibit leukotrienes prior to a trauma to the skin and for a period of time thereafter effectively inhibits the development of hyperpigmentation in people so predisposed.

This application is a continuation of pending provisional application serial No. 60/377,857 filed on May 3, 2002.

### FIELD OF THE INVENTION

The present invention generally relates to substances that inhibit the effects of leukotrienes and more specifically prevent the development of hyperpigmentation after a traumatic event to affected skin.

### BACKGROUND OF THE INVENTION

It is well known that people with certain skin types, i.e., people of Hispanic descent, African descent, Asian descent, and the like, are predisposed to hyperpigementation after a traumatic event to their skin. Such trauma can take the form of bums, abrasions, cuts, surgery, and the like. If the event is predetermined, it can be an additional trauma to the so predisposed individuals. This is particularly true for surgical procedures, laser phototherapy, dermabrasion, and other cosmetic procedures that may traumatize the skin.

To date, the only approach to resultant hyperpigmentation after a traumatic event has been the use of bleaching substances for the skin, such as hydroquinone and related substances, eliminating such exposure and the passage of time. None of these approaches result in uniform success. Nor for that matter, do they give a great deal of satisfaction to the affected individuals. Therefore, there clearly exists a need in the art for a uniformly effective approach to prevent hyperpigmentation in individuals who are so predisposed.

### SUMMARY OF THE INVENTION

It is an object of the present invention to provide a composition, and method related thereto, for the treatment of hyperpigmentation in skin areas.

It is another objection of the present invention to provide a composition, and method related thereto, for the treatment of hyperpigmentation in skin areas, wherein the composition comprises at least one phospholipase A₂ inhibitor, melatonin, menthol, benzyl alcohol, polysorbate 80, and trisoleooxymethylmethylamino-1-ethane sulfonic acid.

It is yet another object of the present invention to provide a composition, and method related thereto, for the treatment of hyperpigmentation in skin areas, wherein the composition comprises at least one phospholipase A₂ inhibitor, melatonin, menthol, benzyl alcohol, polysorbate 80, trisoleooxymethylmethylamino-1-ethane sulfonic acid, propylene glycol, water and mineral oil.

The novel features that are considered characteristic of the invention are set forth with particularity in the appended claims. The invention itself, however, both as to its structure and its operation together with the additional objects and advantages thereof will best be understood from the following description of the preferred embodiment of the present invention. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase. Likewise, the use of the words “function” or “means” in the Description of Preferred Embodiments of the invention is not intended to indicate a desire to invoke the special provision of 35 U.S.C. 112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means of step, then the intention is not to invoke the provisions of 35 U.S.C. §112, paragraph 6. Moreover, even if the provisions of 35 U.S.C. §112, paragraph 6, are invoked to define the inventions, it is intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

### DESCRIPTION OF PREFERRED EMBODIMENTS OF THE INVENTION

The present invention is a composition and method useful for the treatment of hyperpigmentation in skin areas.

Hyperpigmentation, which occurs after trauma to the skin, seems to be mediated by an inflammatory pathway. Probably all individuals exposed to penetrating skin trauma activate this pathway. However, those who are genetically predisposed activate it more substantially than those who are not so predisposed.

The inflammatory pathway involved in the generation of localized excess melanin by melanocytes is apparently mediated by the presence of leukotrienes. Therefore, it is known that such a traumatic event is going to occur, the use of substances that will either competitively inhibit or prevent the formation of leukotrienes will prove useful in preventing subsequent hyperpigmentation. A number of these substances have been used together and separately with surprisingly uniform results.

Surprisingly, for reasons that are not altogether known, leukotriene inhibiting drugs, such as zafirlukas, and others, when taken prior to surgery and for two months or more after surgery, completely prevent the development of hyperpigmentation. Topical administration can also be surprisingly effective. Thus, the ingestion or topical administration of substances that inhibit leukotrienes prior to a trauma to the skin and for a period of time thereafter effectively inhibits the development of hyperpigmentation in people so predisposed.

Arachidonic acid is responsible for the formation of leukotrienes via the lipoxygenase pathway. These substances (leukotrienes) result in a reflex stimulation to melanocytes for the production of melanin. The cycloxygenase pathway, which leads to the production of prostaglandin and thromboxanes, is of no material consequence to this problem since these substances have not been shown to induce hyperpigmentation.

The other system important to the issue of hyperpigmentation is the phospholipase A₂ enzyme system. Although this system is somewhat less specific relative to the production of leukotrienes, it is, nonetheless, important since it is the initial reaction necessary to activate archidonic acid and allow its conversion to leukotrienes.

Clearly substances that block phospholipase A₂ and inhibit leukotriene synthesis would be helpful in the elimination of post-traumatic hyperpigmentation. These substances include, but are not limited to the following leukotriene inhibitors, which are readily available: moneleukast, zafirlukast, zilenton, and others.

Many phospholipase A₂ inhibitors have been defined. Two of the more representative examples are trisoleoyloxy-methylmethylamino-1-ethanaesulfonic acid and corticosteroids. Obviously, corticosteroids, which inhibit all eicosanoid production and therefor result in greatly reduced synthesis of leukotrienes, will be helpful in reducing hyperpigmentation. However, in the case of steroids, the inhibition is not entirely complete and it is well known that all corticosteriods impede wound healing. Hence, these are not ideal substances for use topically or systemically to resolve the problem of post traumatic hyperpigmentation.

To prove the efficacy of this approach, 10 patients, with a strong genetic predisposition to post traumatic hyperpigmentation, were selected for proof of concept. The patients were known to have this predisposition because prior trauma pigmented areas. Five of the subjects were given the oral composition zafirlukast, two weeks prior and thereafter for two months. The dosage was 20 mg two time daily. The other five individuals were give the same medication that the same dosage for the same amount of time, but in addition, were also given a topical preparation. In no case did hyperpigmentation develop. This was found to be quite surprising and novel.

Five further patients were then selected with obvious genetic predisposition to post traumatic hyperpigmentation. They were given only the topical composition after their surgical procedure. The topical composition was applied three times daily for two months after the procedure. Only one individual showed any evidence of hyperpgimentation after the two months period and it was minimal. This was completely unexpected. The topical composition, as administered, is described in Table 1 below.

TABLE 1
Topical composition for the preventing of post traumatic
hyperpigmentation.

| Ingredient | Percentage | Range (%) | Source |
|---|---|---|---|
| Zafirlukast/ | 0.5 | 0.01-90 | Zafirlukast |
| Monteleukast | | | Accolate ™ |
| | | | Astrozeneca |
| | | | Wilmington, Delaware: |
| | | | Monteleukast |
| | | | Singulair ™ |
| | | | Merck |
| | | | White House Station, WS |
| Melatonin | 0.25 | 0.0001-90 | Sigma Chemical |
| | | | St. Louis, MO |
| Propylene glycol | 40 | 0-99 | Sigma Chemical |
| | | | St. Louis, MO |
| Water | 15.5 | 0-99 | |
| Mineral oil | 40 | 0-99 | Sigma Chemical |
| | | | St. Louis, MO |
| Menthol | 0.25 | 0.001-90 | Sigma Chemical |
| | | | St. Louis, MO |
| Benzyl alcohol | 1 | 0.001-90 | Sigma Chemical |
| | | | St. Louis, MO |
| Polysorbate 80 | 0.5 | 0.001-20 | Sigma Chemical |
| | | | St. Louis, MO |
| PX-13 | 0.5 | 0.001-20 | Zenith Cosmetics |
| (Trisoleooxymethyl | | | Aurora, Colorado |
| methylamino-1- | | | |
| ethane sulfonic acid) | | | |

It should be noted that many other phospholipase A₂ inhibitors and leukotriene inhibitors may be used without altering the spirit of the present invention. Melatonin is incorporated in the topical formula because it is a known melanoncyte stimulating hormone antagonist.

The preferred embodiment of the invention is described above in the Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

What is claimed is:

1. A leukotriene inhibiting composition comprising at least one phospholipase A₂ inhibitor, melatonin, menthol, benzyl alcohol, polysorbate 80, and trisoleooxymethylmethylamino-1-ethane sulfonic acid.

2. The leukotriene inhibiting composition according to claim 1 wherein
a. the at least one phospholipase A₂ inhibitor ranges from 0.01-90% by weight;
b. the melatonin ranges from 0.0001-90% by weight;
c. the menthol ranges from 0.001-90% by weight;
d. the benzyl alcohol ranges from 0.001-90% by weight;
e. the polysorbate 80 ranges from 0.001-20% by weight; and
f. the trisoleooxymethylmethylamino-1-ethane sulfonic acid ranges from 0.001-20% by weight.

3. The leukotriene inhibiting composition according to claim 2 wherein
a. the at least one phospholipase A₂ inhibitor is approximately 0.5% by weight;
b. the melatonin is approximately 0.25% by weight;
c. the menthol is approximately 0.25% by weight;
d. the benzyl alcohol is approximately 1% by weight;
e. the polysorbate 80 is approximately 0.5% by weight; and
f. the trisoleooxymethylmethylamino-1-ethane sulfonic acid is approximately 0.5% by weight.

4. The leukotriene inhibiting composition according to claim 2 further comprising:
a. propylene glycol ranging from 0-99% by weight;
b. water ranging from 0-99% by weight; and
c. mineral oil ranging from 0-99% by weight.

5. The leukotriene inhibiting composition according to claim 3 further comprising:
a. propylene glycol ranging from 0-99% by weight;
b. water ranging from 0-99% by weight; and
c. mineral oil ranging from 0-99% by weight.

6. The leukotriene inhibiting composition according to claim 4 further comprising:
a. propylene glycol is approximately 40% by weight;
b. water is approximately 15.5% by weight; and
c. mineral oil is approximately 40% by weight.

7. The leukotriene inhibiting composition according to claim 5 further comprising:
a. propylene glycol is approximately 40% by weight;
b. water is approximately 15.5% by weight; and
c. mineral oil is approximately 40% by weight.

8. A method for inhibiting leukotriene production comprising the step of applying composition comprising at least one phospholipase A₂ inhibitor, melatonin, menthol, benzyl alcohol, polysorbate 80, and trisoleooxymethylmethylamino-1-ethane sulfonic acid to a skin surface.

9. The method according to claim 8 wherein the composition further comprises
a. the at least one phospholipase A₂ inhibitor ranges from 0.01-90% by weight;
b. the melatonin ranges from 0.0001-90% by weight;
c. the menthol ranges from 0.001-90% by weight;
d. the benzyl alcohol ranges from 0.001-90% by weight;
e. the polysorbate 80 ranges from 0.001-20% by weight; and
f. the trisoleooxymethylmethylamino-1-ethane sulfonic acid ranges from 0.001-20% by weight.

10. The method according to claim 9 wherein the composition further comprises
a. the at least one phospholipase A₂ inhibitor is approximately 0.5% by weight;
b. the melatonin is approximately 0.25% by weight;
c. the menthol is approximately 0.25% by weight;
d. the benzyl alcohol is approximately 1% by weight;
e. the polysorbate 80 is approximately 0.5% by weight; and
f. the trisoleooxymethylmethylamino-1-ethane sulfonic acid is approximately 0.5% by weight.

11. The method according to claim 9 wherein the composition further comprises
a. propylene glycol ranging from 0-99% by weight;
b. water ranging from 0-99% by weight; and
c. mineral oil ranging from 0-99% by weight.

12. The method according to claim 10 wherein the composition further comprises
a. propylene glycol ranging from 0-99% by weight;
b. water ranging from 0-99% by weight; and
c. mineral oil ranging from 0-99% by weight.

13. The method according to claim 1 wherein the composition further comprises
a. propylene glycol is approximately 40% by weight;
b. water is approximately 15.5% by weight; and
c. mineral oil is approximately 40% by weight.

14. The method according to claim 12 wherein the composition further comprises
a. propylene glycol is approximately 40% by weight;
b. water is approximately 15.5% by weight; and
c. mineral oil is approximately 40% by weight.
